(19) United States
(12) Patent Application Publication
(10) Pub. No.: US 2006/0137683 A1
(43) Pub. Date: Jun. 29, 2006

(54) Nozzle

(76) Inventors: Gregor John McLennan Anderson, Ware (GB); David Edgerley, London (GB); Robert Edward Matthews, London (GB)

Correspondence Address: GLAXOSMITHKLINE;CORPORATE INTELLECTUAL PROPERTY, MAI B475, FIVE MOORE DR., PO BOX 13398, RESEARCH TRIANGLE PARK, NC, 27709-3398, US

(21) Appl. No.: 10/559,257

(22) PCT Filed: Jun. 10, 2004
(86) PCT No.: PCT/GB04/02443

§ 371 Date: Dec. 1, 2005

(30) Foreign Application Priority Data
Jun. 10, 2003 (GB) 0313355.0

(51) Int. Cl. A61M 11/00; A62B 7/10

(52) U.S. Cl. 128/200.21; 128/200.14; 128/206.11; 128/207.18

(57) ABSTRACT

A shark fin like shaped nozzle for an intranasal dispenser is disclosed. The nozzle has a tear drop or lozenge shaped section through its (in use) horizontal plane and a generally rearwardly directed vent for dispensing medicament directly into an inner nasal space of a user. A cap is attached to the front of the nozzle by a hinge for enclosing the nozzle and for preventing the incorrect application of the nozzle into a nostril.

### FIELD OF THE INVENTION

The present invention relates to a nozzle for an intranasal dispenser.

### BACKGROUND OF THE INVENTION

Intranasal dispensers are well known for dispensing liquid, gas or powdered medicaments to a patient or user via the user's nostril. Dispensers generally comprise a bottle having a nozzle on a cap of the bottle. The nozzles, therefore, are generally provided at the top of the dispensers. Nozzles generally comprise a generally cylindrical tube. The bottle can be squeezed to dispense a measure or dose of the contents of the bottle through the nozzle. Alternatively, the dispenser may have a pump or valve to dispense through the nozzle.

The nozzle, which will be inserted into a nostril, will therefore guide the medicament into the nasal passage and ultimately, perhaps by inhaling, into the inner nasal space.

FIG. 1 shows a typical, generally cylindrical, nozzle 1 of a prior art intranasal dispenser. The nozzle 1 has a vent 3 at the top of it to direct medicament in an axial direction 5 relative to the nozzle 1 when dispensing the medicament.

In use, the nozzle 1 is inserted into a nostril so that the medicament to be dispensed can be directed into the nose and delivered into the inner nasal space behind the nose.

FIG. 2 shows the inner nasal space 6 of a typical human user to which access is gained through the nostrils 7 in the nose 8. Ideally, administered medicament should be dispensed deep into the inner nasal space (i.e. towards the rear or left-hand side as viewed in FIG. 2), and preferably onto the nasal fossae (not shown). However, users commonly have a natural phobia against inserting a nozzle deep into the nose.

As an example, FIG. 3A shows how the prior art nozzle 1 of FIG. 1 would need to be angled backwards in the nostril 7 of a human user so that the dispensing path 5 is oriented optimally towards the back of the inner nasal space 6. However, studies have shown that this orientation of the nozzle 1 is unpleasant for the user. Accordingly, the user will direct the nozzle 1 as shown in FIG. 3B, resulting in the medicament being dispensed upwardly into the front region of the inner nasal space 6.

Therefore, the nozzle 1 of the prior art is inherently non-optimal since to dispense the medicament correctly, a user will need to face his or her phobia or will need to inhale sharply. Inhaling sharply, however, can cause sneezing, thereby expelling the administered medicament.

It would therefore be desirable to provide a new nozzle design for intranasal dispensers that will provide a more efficient dispensing of a medicament into the inner nasal space.

### SUMMARY OF THE INVENTION

The present invention provides a nozzle for an intranasal dispenser having a generally shark fin like shape, i.e. like a dorsal fin. This shape allows the nozzle to fit accurately within a nostril and prevents the nozzle from being inserted too deeply into the nostril.

The present invention also provides a nozzle for an intranasal dispenser having an elongated shape in transverse section, i.e. through its, in use, horizontal plane. Whereas prior art nozzles are generally cylindrical in transverse cross-section, FIGS. 4 to 4D show that human (and other animal) nostrils 7 are generally elongated in transverse cross section. The section of the present invention, therefore, correctly fits and orientates itself in a nostril.

The present invention also provides a nozzle for an intranasal dispenser having a vent for dispensing medicament therefrom directed non-parallel to the longitudinal axis of the nozzle. Preferably, this orientation of the vent is combined with providing the nozzle with the elongated shape and/or the shark fin shape. Preferably the orientation is such that the vent's axis extends in the plane of symmetry of the nozzle, or rearwardly relative to the nostril into which the nozzle will, in use, be inserted. Orienting the nozzle in this manner results in a medicament being dispensed directly towards the inner nasal space.

Preferably the nozzle is fitted to an intranasal dispenser. Preferably the intranasal dispenser has a cap thereon for enclosing the nozzle.

The present invention further provides an intranasal dispenser having a nozzle according to the invention.

The present invention also provides an intranasal dispenser comprising a nozzle having a vent angled in a first direction relative to the longitudinal axis of the nozzle and a cap for the nozzle hinged relative to the nozzle to open away from that first direction.

Further preferred features of the present invention are set forth in the claims appended hereto, as well as in the non-limiting exemplary embodiments of the invention which will now be described with reference to the accompanying Figures of drawings.

### BRIEF DESCRIPTION OF THE FIGURES OF DRAWINGS

FIG. 1 shows a prior art nozzle design;

FIG. 2 shows a vertical section through a nose and inner nasal space taken perpendicular to a face of a human subject;

FIG. 3A shows a use of the prior art nozzle that would be deemed unpleasant by a human user;

FIG. 3B shows a use of the prior art nozzle that would be deemed acceptable by a user;

FIG. 4 shows a partial section through a pair of human nostrils viewed from below;

FIGS. 4A to 4D show various further horizontal sections of human nostrils;

FIG. 5 shows a first nozzle of the present invention on an intranasal dispenser;

FIG. 6 is a perspective view of a second nozzle of the present invention;

FIG. 7 is a side view of the second nozzle showing it dispensing with a cloud dispersal pattern;

FIG. 7A is a plan view of the second nozzle;

FIG. 8 shows the second nozzle of the present invention modified to dispense with a jet dispersal pattern;

FIG. 9 shows a rear perspective view of the second nozzle; and

FIG. 10 is scrap, longitudinal cross-sectional view of the second nozzle.

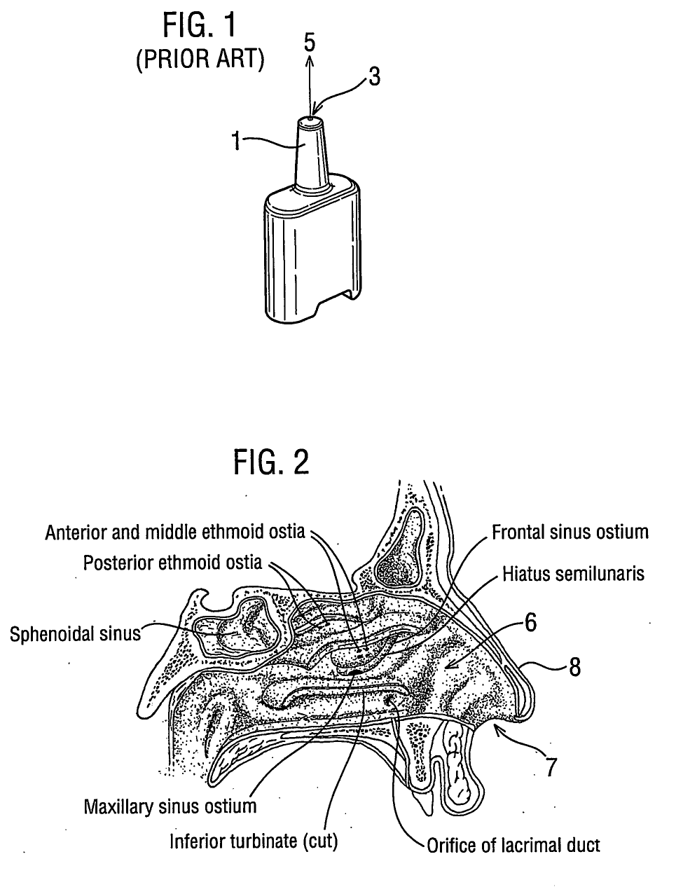

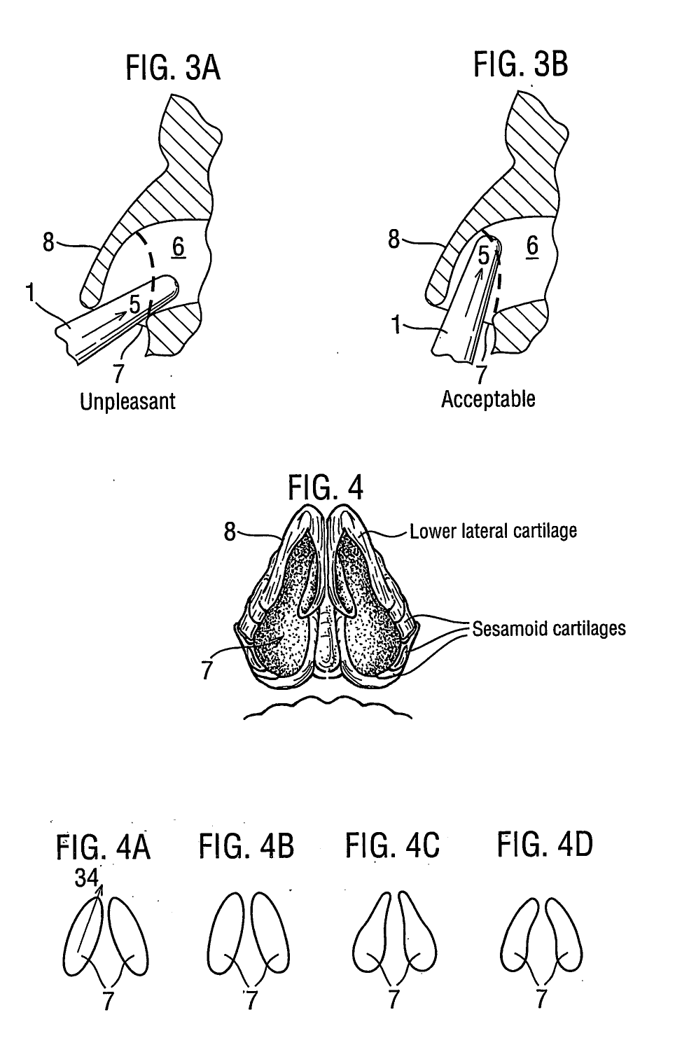

### DETAILED DESCRIPTION OF THE FIGURES OF DRAWINGS

In the following description like features of the different embodiments are assigned like reference numerals.

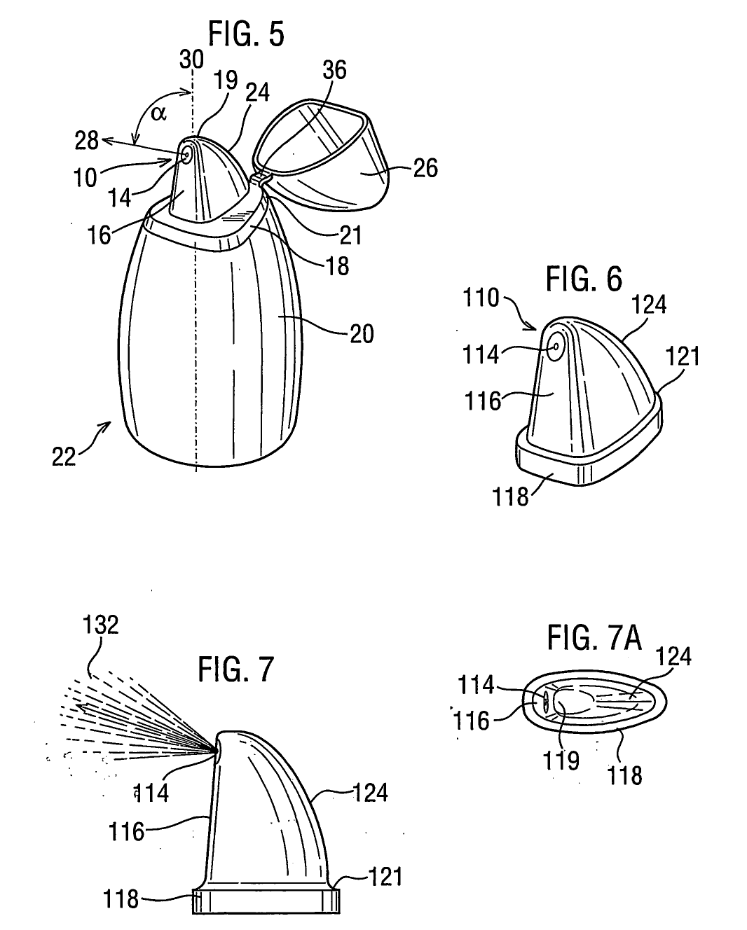

In FIG. 5 there is shown a first nozzle 10 according to the present invention. The nozzle 10 has, in longitudinal cross section, a generally shark fin like shape. In transverse cross section, the nozzle 10 has a generally tear drop or lozenge like shape corresponding generally with the shape of a nostril in transverse section, i.e. horizontal in use, as shown in FIG. 4A. This shape allows the nozzle 10 to be inserted into a nostril only in a correct (and comfortable) orientation. This profile of the nozzle 10 will be further understood by reference to FIGS. 6-10 which illustrate a second nozzle 110 of the invention of corresponding longitudinal and transverse cross-sectional shape.

The tear drop section of the nozzle 10 has a long axis 34 (see FIG. 4A). The shark fin like shape has a longitudinal axis 30. The long axis 34 and the longitudinal axis 30 lie within a plane of symmetry of the nozzle 10.

The nozzle 10 comprises a vent 14 through which medicament can be dispensed. The vent 14 has a medicament dispensing axis 28 that extends generally rearwardly relative to a nostril into which the nozzle 10 will be inserted. The dispensing axis 28 lies in the plane of symmetry of the nozzle 10. In this embodiment the dispensing axis 28 defines an angle α of about 70° relative to the longitudinal axis 30 of the nozzle 10 and generally towards the long axis 34. However, the angle α may be anywhere from 100° to 55°.

The shark fin like shape of the nozzle 10 has a rear surface 16 that extends substantially perpendicularly from a base 18 of the nozzle 10. The vent 14 is provided on this rear surface 16, towards a top or tip 19 thereof.

The base 18 of the nozzle is adapted to fit onto a fluid container 20 or bottle of an intranasal dispenser 22 containing a fluid medicament, typically a liquid.

A convexly curved front surface 24 opposes the rear surface 16 of the nozzle 10. This front surface 24 arches from the tip 19 of the shark fin, i.e. at or near the top of the rear surface 16 of the nozzle, forwardly and downwards therefrom towards a front 21 of the base 18. The front surface 24 of the nozzle, in use, will rest against the fleshy, internal surface of the nostril.

The sides of the nozzle 10, and any junctions between them, are also convexly curved so that the nozzle 10 has a smooth finish; it should be comfortable when inserted into a nostril.

A cap 26 is attached to the base 18 of the nozzle 10 by a hinge 36, preferably a living hinge. The cap 26 can be folded over the nozzle 10 to enclose the nozzle 10 when the intranasal dispenser 22 is not in use. Instead of a hinge 36, the cap 26 could be screw fitted onto the base 18 of the nozzle 10 or it could be snap fitted thereover. Screw connections and snap fittable connections are well known in the art. However, the hinged connection has an advantage.

The hinge 36 is positioned adjacent the front 21 of the base 18, i.e. where the front surface 24 of the nozzle 10 joins the base 18. Positioning the hinge 36 here ensures that the cap 26, when folded into the open position shown in FIG. 5, will not interfere with the insertion of the nozzle 10 into a nostril. Further, its position ensures the nozzle is inserted into the nostril at a correct orientation, i.e. not backwards. The cap 26, clearly, must be positioned away from the face of the user in order for the nozzle 10 to be inserted into a nostril.

It will be appreciated from the above description that the orientation of the vent 14 is such as to dispense medicament from the dispenser 22 rearwardly or away from the cap 26. Further, it will be appreciated that the dispensing is not parallel to the longitudinal axis 30 of the nozzle. Therefore, in use, medicament will be able to be dispensed by the nozzle 10 directly towards the back of the inner nasal space, preferably at the nasal fossae, to optimise the effectiveness of the medicament.

In FIGS. 6-10 there is shown a second nozzle 110 of the present invention which corresponds to the first nozzle 10 other than not having the hinged cap 26. Thus, the second nozzle 110 has inter alia the same longitudinal and transverse cross-sectional profiles as detailed for the nozzle 10 of FIG. 5, and the other common features will be self-evident from the drawings and the like reference numerals. Moreover, the second nozzle 110 is usable with the container 20 shown in FIG. 5 to form the intranasal dispenser 22.

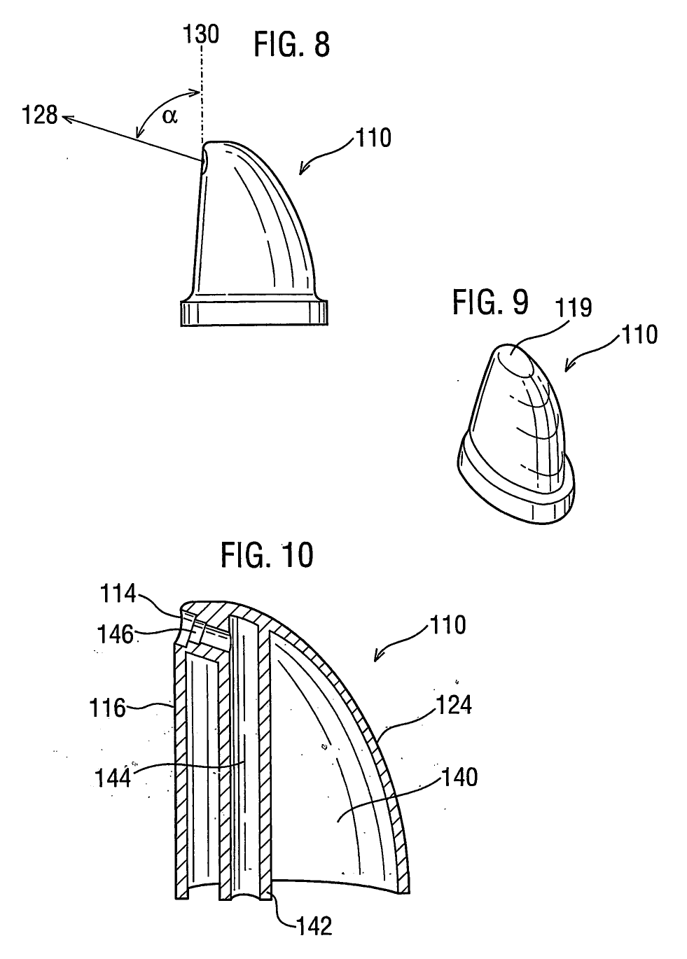

In FIG. 7 the nozzle 110 is shown having a vent 114 having a cloud dispersal pattern 132 falling generally within the range of angles given above for the first nozzle 10. FIG. 8 shows the nozzle 110 having a vent 114 having a jet dispersal pattern 128 at an angle α of about 70°, although the previously disclosed angle range would apply equally for the jet dispersal pattern 128. A person skilled in the art of nozzle design will readily be able to provide either cloud or jet dispersal patterns of these types.

FIG. 10 shows that the second nozzle 110 has a hollow interior 140 in which is formed a conduit structure 142 through which the medicament in the container 20 is able to be discharged from the intranasal dispenser 22 via the nozzle 110. More particularly, the conduit structure 142 comprises a longitudinal section 144 and a transverse section 146 extending between, and in fluid communication with, the longitudinal section 144 and the vent 114. The longitudinal section 144 couples with the outlet (not shown) of the container 20, which may be provided by a pump or valve, as will be understood by the skilled reader in the art.

It will be understood that the embodiments of the present invention described above are purely by way of example, modifications and variations being able to be made within the scope of the invention as defined by the claims appended hereto.

The present application claims priority from UK patent application No. 0 313 355.0 filed on 10 Jun. 2003, the entire content of which is hereby incorporated herein by reference.

1. A nozzle for an intranasal dispenser having a generally shark fin like shape.

2. A nozzle for an intranasal dispenser having an elongated shape in transverse section.

3. The nozzle of claim 2 having a generally shark fin like shape.

4. A nozzle for an intranasal dispenser having a vent for dispensing medicament therefrom directed non-parallel to the longitudinal axis of the nozzle.

5. The nozzle of claim 4 having a generally shark fin like shape.

6. The nozzle of claims 1 or 2, wherein a vent for dispensing medicament therefrom is oriented such that the vent's axis extends generally rearward relative to the nostril into which the nozzle will, in use, be inserted.

7. The nozzle of claims 1 or 2, comprising a convex forward surface and a generally straight rear surface, the rear surface having a vent therein for dispensing medicament.

8. The nozzle of claim 7, wherein the rear surface extends substantially parallel to the longitudinal axis of the nozzle and perpendicular to a base of the nozzle.

9. The nozzle of claim 7, wherein the vent is adapted to dispense medicament at an angle of about 70° relative to the longitudinal axis of the nozzle and substantially rearward of the rear surface.

10. (canceled)

11. The nozzle of claims 1, 2 or 4, having a cap for enclosing the nozzle.

12. An intranasal dispenser having a nozzle according to claims 1, 2 or 4.

13. An intranasal dispenser comprising a nozzle having a vent angled in a first direction relative to the longitudinal axis of the nozzle or dispenser and a cap for the nozzle hinged relative to the nozzle to open away from that first direction.

14. The dispenser of claim 13, wherein the nozzle is in accordance with claims 1, 2 or 4.

15. (canceled)

16. (canceled)

17. (canceled)
